(19) United States
(12) Patent Application Publication
(10) Pub. No.: US 2022/0168488 A1
(43) Pub. Date: Jun. 2, 2022

(54) Double-Lumen Percutaneous Femoral Cannula for Extracorporeal Membrane Oxygenation

(71) Applicant: Kok Hoo Lim, Beavercreek, OH (US)

(72) Inventor: Kok Hoo Lim, Beavercreek, OH (US)

(21) Appl. No.: 17/538,595

(22) Filed: Nov. 30, 2021

Related U.S. Application Data
(60) Provisional application No. 63/119,493, filed on Nov. 30, 2020.

(51) Int. Cl. A61M 1/36; A61M 25/00; A61M 25/01

(52) U.S. Cl. CPC A61M 1/3659; A61M 1/1698; A61M 25/0108; A61M 25/003

(57) ABSTRACT

A double-lumen percutaneous femoral cannula for extracorporeal membrane oxygenation (ECMO) is an apparatus that is used to perform an ECMO procedure in non-ideal conditions. The apparatus may include at least one first cannula and at least one second cannula of different sizes that are arranged to form a single structure. By providing a single structure instead of several separate components, the apparatus makes the insertion of the apparatus easier and reduces the risk of the apparatus dislodging while the patient is being transported. The at least one first cannula and the at least one second cannula are preferably elongated tubular structures designed to prevent the recirculation of oxygenated blood. The at least one first cannula is designed to enable the aspiration of deoxygenated blood, while the at least one second cannula is designed to enable the pumping of oxygenated blood back into the body.

The current application claims a priority to the U.S. provisional patent application Ser. No. 63/119,493 filed on Nov. 30, 2020.

### FIELD OF THE INVENTION

The present invention relates generally to medical and laboratory equipment. More specifically, the present invention is a double lumen percutaneous femoral cannula designed to facilitate the procedure for extracorporeal membrane oxygenation (ECMO).

### BACKGROUND OF THE INVENTION

Extracorporeal membrane oxygenation (ECMO) may be the last resort for patients with acute respiratory failure who do not respond to conventional ventilator therapy and medical management. There are many possible causes of acute respiratory failure. Today with the pandemic, severe cases of COVID-19 are the leading cause of acute respiratory failure. During the ECMO procedure, deoxygenated blood is drained from the vena cava or right atrium into a membrane oxygenator outside the body. Oxygen is then exchanged for carbon dioxide through an artificial membrane in the membrane oxygenator. The oxygenated blood is then circulated through a centrifugal pump where the blood is pressurized and then pumped back into the right atrium.

There are different methods to perform an ECMO. In a veno-arterial ECMO, the oxygenated blood is pumped into the arterial system. Alternatively, the patient may be cannulated for a veno-veno ECMO through three options. A first option may include an internal jugular vein-femoral vein. Under normal circumstances, when the patients can be positioned properly, inserting a catheter into the internal jugular vein is straight forward. Deoxygenated blood is drained from the femoral vein catheter that may advance into an inferior vena cava, blood is oxygenated in membrane oxygenation, and then pumped back into the patient through the catheter in the internal jugular vein. However, for a patient with respiratory failure on a roto-prone bed, the patient may be too ill to be brought to the operating room.

The procedure associated with the insertion of the catheter into the internal jugular vein may also be done by the bedside in the intensive care unit. However, there may be different situations that makes the insertion procedure more complicated. For example, the patient may be on a ventilator, on high positive pressure, and 100% inhaled oxygen. Further, the patient may be strapped onto the bed, and it is difficult to turn the head sideways to facilitate insertion of the needle into the internal jugular vein. The head of the bed needs to be elevated to optimize oxygenation, but the patients may not tolerate being placed in a supine position. Inserting a cannula into the internal jugular vein under these circumstances is technically challenging. Adding to the difficulty, fluoroscopy may not be used while the patient is on the roto-prone bed. Furthermore, transesophageal echocardiography (TEE) may not be available to assist in advancing a large cannula from the internal jugular vein into the right atrium. Cannulation of the femoral vein and advancing the cannula into an inferior vena cava is relatively straight forward. With the right internal jugular vein and the femoral vein configuration of veno-veno ECMO, when the patient is moved from the roto-prone bed to the transport stretcher during transportation or regular turning of position of the patients in the ICU, the right internal jugular vein catheter may get misplaced. Worse, the catheter may get dislodged from the internal jugular vein, causing an immediate disaster.

A second method may include a double-lumen catheter for veno-veno ECMO placed in the internal jugular vein. For this method to work, meaning achieving a flow of 3 to 5 liters per minute, the size of the double-lumen catheter must be at least 27 French (Fr) to 31 Fr. Like the first option, this catheter must be inserted under suboptimal conditions. Without the fluoroscopy or the TEE to guide the insertion, there is a risk of a stiff guide wire causing perforation of the right atrium or ventricle. Further, a large size double lumen cannula is known to cause injury to the superior vena cava, the right atrium, and right ventricle even under the best circumstances. Another disadvantage of the second option is the increase in recirculation of the oxygenated and deoxygenated blood because the distance between the inflow and outflow is short, which reduces the effectiveness of the veno-veno ECMO.

Furthermore, a third method of the three options may include cannulation of bilateral femoral vein. The third option may be preferred by some institutions when the patient with acute respiratory failure on the ECMO is to be transferred from another outlying hospital. A venous cannula is inserted into each femoral vein of the patient. One of the cannulas is advanced into the right atrium to serve as inflow for oxygenated blood to be injected into the right atrium. The second cannula remain in the inferior vena cava to serve as outflow, draining the deoxygenated blood into the oxygenator. However, this third method also requires almost ideal conditions to avoid complications during the ECMO procedure. Therefore, there is a need for an improved double-lumen percutaneous femoral cannula for ECMO that may overcome one or more of the above-mentioned problems and/or limitations.

An objective of the present invention is to provide a double-lumen percutaneous femoral cannula for ECMO that is designed to prevent the recirculation of the oxygenated blood and the deoxygenated blood during the ECMO procedure. Another objective of the present invention is to provide a double-lumen percutaneous femoral cannula for ECMO that can be securely inserted into the body of a patient being transported without risk of the present invention dislodging. Another objective of the present invention is to provide a double-lumen percutaneous femoral cannula for ECMO that can be inserted in non-ideal conditions without high risk of damage to the patient. Additional features and benefits of the present invention are further discussed in the sections below.

### SUMMARY OF THE INVENTION

The present invention is a double-lumen percutaneous femoral cannula for veno-veno extracorporeal membrane oxygenation (VV-ECMO). The present invention includes a pair of cannulas of different dimeters, with the diametrically smaller cannula positioned within the diametrically larger cannula. The diametrically larger cannula is preferably used to aspirate deoxygenated blood, while the diametrically smaller cannula is used to pump in oxygenated blood. The nested configuration of the pair of cannulas facilitates the insertion of the present invention for the VV-ECMO procedure and prevents the dislodgement of either cannula if the patient is being transported. In addition, the present invention is designed to prevent the recirculation of the oxygenated blood being pumped in via the diametrically smaller cannula.

### BRIEF DESCRIPTION OF THE DRAWINGS

FIG. 1 is a top front perspective view of the present invention.

FIG. 2 is a magnified view of the second cannula outlet of the present invention.

FIG. 3 is a top rear perspective view of the present invention.

FIG. 4 is a magnified view of the first cannula inlet of the present invention.

FIG. 5 is a front view of the present invention.

FIG. 6 is an enlarged horizontal cross-sectional view taken in the direction of line 6-6 in FIG. 5.

FIG. 7 is a side view of the present invention.

FIG. 8 is a vertical cross-sectional view taken in the direction of line 8-8 in FIG. 7.

FIG. 9 is a magnified view of the plurality of first corrugated rings and the plurality of second corrugated rings of the present invention.

FIG. 10 is a schematic view of the present invention, wherein the present invention is shown utilized in a veno-veno ECMO configuration.

### DETAIL DESCRIPTIONS OF THE INVENTION

All illustrations of the drawings are for the purpose of describing selected versions of the present invention and are not intended to limit the scope of the present invention.

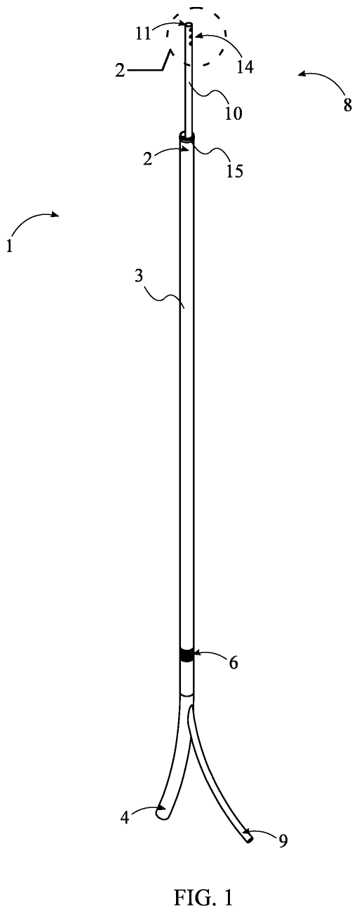

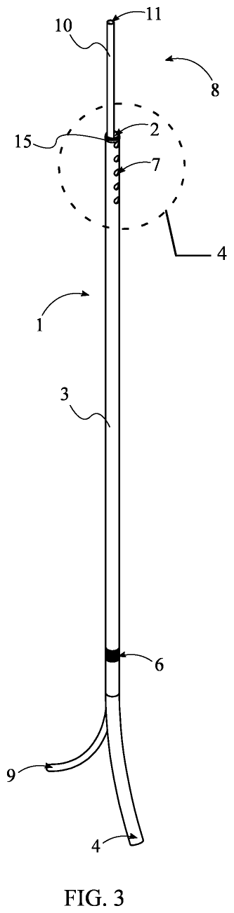

The present invention is a double-lumen percutaneous femoral cannula for extracorporeal membrane oxygenation (ECMO) that is easier to insert even in non-ideal conditions and does not dislodge easily while the patient is being transported. While many aspects and features relate to, and are described in the context of, a double-lumen percutaneous femoral cannula for extracorporeal membrane oxygenation, different embodiments of the present invention are not limited to use only in this context. As can be seen in FIGS. 1, 3, 5, and 10, the present invention may comprise at least one first cannula 1 and at least one second cannula 8 of different diameters that are arranged to form a single structure. By providing a single structure instead of several separate components, the present invention makes the ECMO procedure go smoother without risking damage to the patient even in non-ideal conditions.

The general configuration of the aforementioned components enables medical staff to easily insert a cannula structure in order to perform an ECMO procedure. As can be seen in FIGS. 1, 3, 5, and 10, the at least one first cannula 1 and the at least one second cannula 8 are preferably elongated tubular structures that enable the inflow of oxygenated blood and the outflow of deoxygenated blood. So, the at least one first cannula 1 comprises a first cannula inlet 2, a first cannula body 3, and a first cannula outlet 4. The first cannula inlet 2 and the first cannula outlet 4 preferably correspond to the terminal open ends of the first cannula body 3. Likewise, the at least one second cannula 8 comprises a second cannula inlet 9, a second cannula body 10, and a second cannula outlet 11. The second cannula inlet 9 and the second cannula outlet 11 preferably correspond to the terminal open ends of the second cannula body 10.

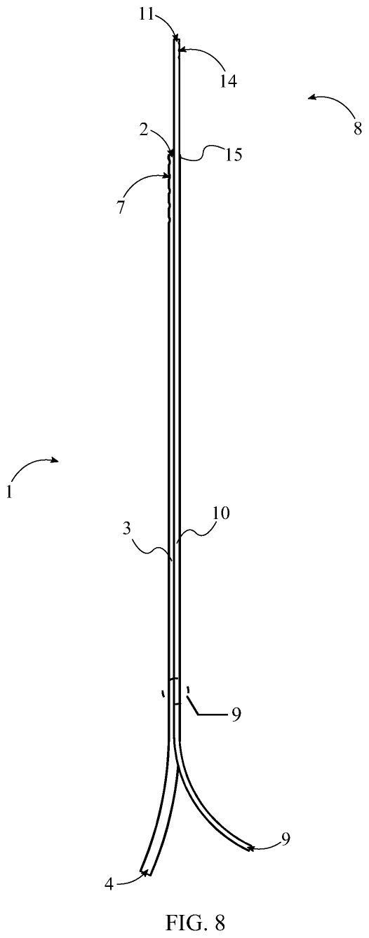

To form a single tubular structure, the second cannula inlet 9 laterally and hermetically traverses into the first cannula body 3, adjacent to the first cannula outlet 4. As can be seen in FIGS. 1, 3, 5, 8, and 10, the second cannula body 10 also traverses through the first cannula body 3 and out of the first cannula inlet 2. This arrangement of the first cannula body 3 and the second cannula body 10 keeps the blood flow through the different cannula bodies separate from each other. In addition, the second cannula body 10 is tangentially mounted within the first cannula body 3 to prevent the second cannula body 10 from moving within the first cannula body 3 which may obstruct the blood flow through the first cannula body 3. Furthermore, to prevent the recirculation of the blood flow being pumped in via the first cannula body 3, the second cannula outlet 11 is positioned external to the first cannula body 3, offset to the first cannula inlet 2. The second cannula outlet 11 may be position at least 10 centimeters (cm) apart from the first cannula inlet 2. The distance between the first cannula inlet 2 and the second cannula outlet 11 reduces the recirculation of the oxygenated blood and the deoxygenated blood, which improves the efficiency of the veno-veno ECMO. Further, the Eustachian valve present between the inferior vena cava and the right atrium helps reduce the mixing of the oxygenated blood that has injected into the right atrium with the deoxygenated blood in the inferior vena cava.

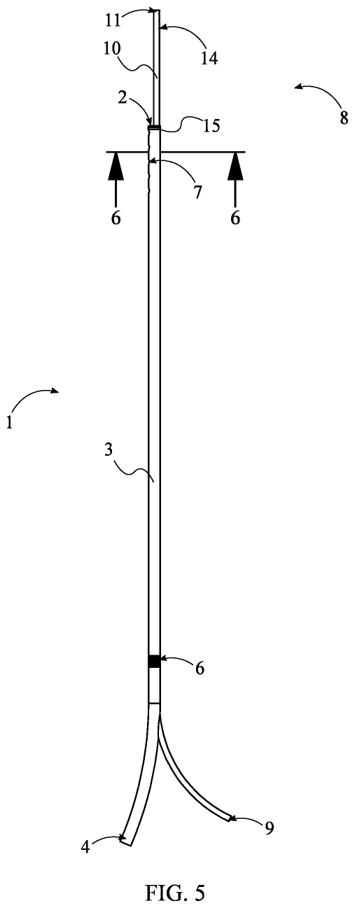

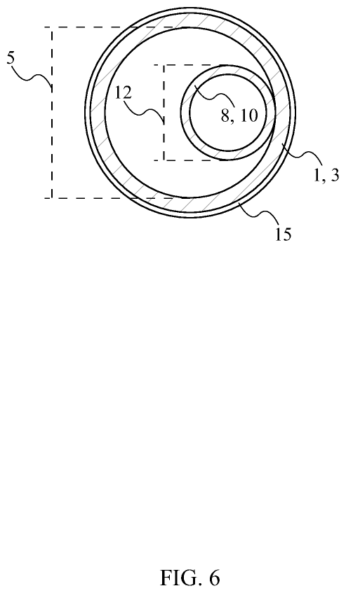

To ensure that the blood flow through the first cannula body 3 is not obstructed by the second cannula body 10, an inner diameter 5 of the first cannula body 3 is larger than an outer diameter 12 of the second cannula body 10, as can be seen in FIGS. 5 and 6. Thus, the blood flows through the two cannula bodies are kept separate from each other and unobstructed. Furthermore, to enable the connection of the cannulas to the corresponding sources, the first cannula outlet 4 and the second cannula inlet 9 are oriented away from each other. This enables the medical staff to connect the first cannula outlet 4 to the inlet of the oxygenation mechanism and the second cannula inlet 9 to the outlet of the oxygenation mechanism.

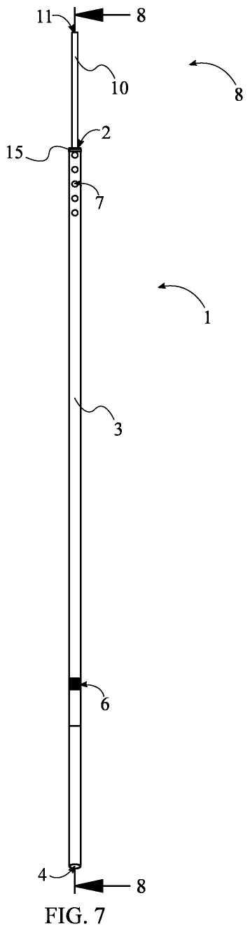

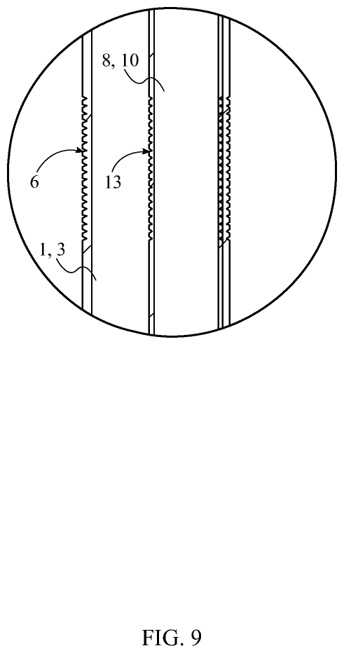

To further facilitate the insertion and the removal of the present invention without damaging any internal organs, the at least one first cannula 1 may further comprise a plurality of first corrugated rings 6. As can be seen in FIG. 7 through 9, the plurality of first corrugated rings 6 enables the at least one first cannula 1 to be bent and twisted to facilitate the insertion and removal of the present invention. To do so, the plurality of first corrugated rings 6 is laterally integrated into the first cannula body 3. In addition, the plurality of first corrugated rings 6 is positioned offset from the first cannula inlet 2 and offset from the first cannula outlet 4. This positions the plurality of first corrugated rings 6 in a central position along the first cannula body 3 that enables the first cannula body 3 to be as flexible as possible.

In addition, to match the flexibility of the at least one first cannula 1, the at least one second cannula 8 may further comprise a plurality of second corrugated rings 13. As can be seen in FIG. 7 through 9, the plurality of second corrugated rings 13 enables the at least one second cannula 8 to twist and bend according to the movements of the at least one first cannula 1. To do so, the plurality of second corrugated rings 13 is laterally integrated into the second cannula body 10. In addition, the plurality of second corrugated rings 13 is positioned offset from the second cannula inlet 9 and offset from the second cannula outlet 11. Further, the plurality of second corrugated rings 13 is positioned within the first cannula body 3, adjacent to the plurality of first corrugated rings 6. This ensures that the positioning of the plurality of second corrugated rings 13 matches the positioning of the plurality of first corrugated rings 6. Thus, as the first cannula body 3 is bent or twisted, the second cannula body 10 matches the movement in order to protect the second cannula body 10 from damage.

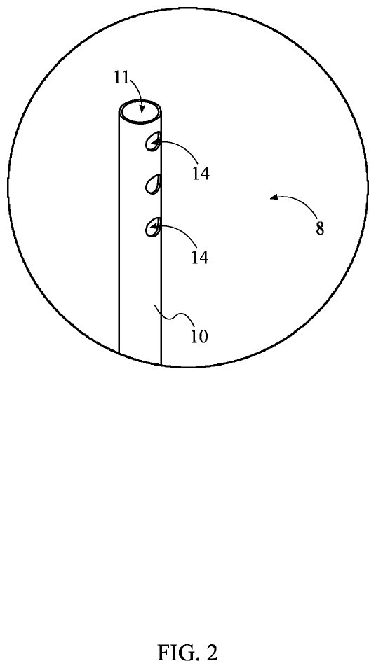

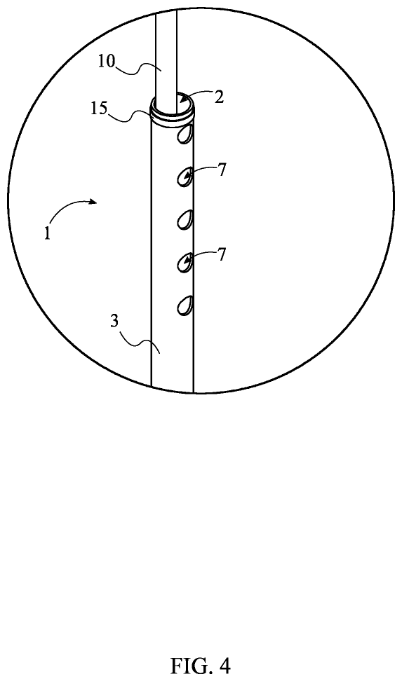

As previously discussed, the present invention enables the aspiration of deoxygenated blood and pumping of oxygenated blood. As can be seen in FIG. 1 through 4, to facilitate the aspiration of deoxygenated blood, the at least one first cannula 1 may further comprise a plurality of first orifices 7. The plurality of first orifices 7 increases the amount of deoxygenated blood entering the first cannula body 3. To do so, the plurality of first orifices 7 laterally traverse into the first cannula body 3, adjacent to the first cannula inlet 2. The plurality of first orifices 7 is also positioned offset from the second cannula body 10 about the first cannula body 3. This ensures that the plurality of first orifices 7 increases the volumetric outflow of deoxygenated blood via the first cannula body 3.

Similar to the plurality of first orifices 7, the at least one second cannula 8 may further comprise a plurality of second orifices 14. As can be seen in FIG. 1 through 4, the plurality of second orifices 14 increases the outflow of oxygenated blood flowing through the second cannula body 10. To do so, the plurality of second orifices 14 laterally traverses into the second cannula body 10, adjacent to the second cannula outlet 11. In addition, the plurality of second orifices 14 is positioned external to the first cannula body 3. This ensures that the plurality of second orifices 14 increases the volumetric outflow of oxygenated blood via the second cannula body 10. In addition, the positioning of the plurality of first orifices 7 and the plurality of second orifices 14 further prevents the recirculation of oxygenated blood by orienting the outflow of deoxygenated blood and the inflow of oxygenated blood opposite and apart from each other.

In a preferred embodiment, the first cannula body 3 and the second cannula body 10 may be made of silastic material, with an overall length of 70 cm. The outer diameter of the first cannula body 3 is preferably 27 Fr to 33 Fr. The outer diameter 12 of the second cannula body 10 is 12 Fr to 15 Fr. The plurality of first orifices 7 preferably includes five first orifices to allow the deoxygenated blood to drain from the inferior vena cava to the membrane oxygenator. Carbon dioxide from the venous blood is exchanged for oxygen in the membrane oxygenator. The oxygenated blood is then circulated around the centrifugal pump where the oxygenated blood is pressurized and pump back into the patient through the at least one second cannula 8. The plurality of second holes preferably includes three holes that are directed across the opening of the tricuspid valve. Further, a tubing that connects the patient to the membrane oxygenator may be of a diameter of 0.75 inches (in). Two connectors may be required to adapt to the shape and size of the first cannula outlet 4 and the second cannula inlet 9. The first cannula outlet 4 may be labeled with a “V” for a venous connector to be used for the at least one first cannula 1. The second cannula inlet 9 may be labeled with an “A” for an arterial connector to be used with the at least one second cannula 8.

Furthermore, to facilitate the insertion of the present invention, the present invention may further comprise a radiopaque marker 15. As can be seen in FIG. 1 through 4 and 10, the radiopaque marker 15 facilitates the insertion of the present invention by enabling the medical staff to track the position of the present invention using an X-ray machine or similar mechanism. To do so, the radiopaque marker 15 is integrated into the first cannula body 3, adjacent to the first cannula inlet 2. This way, the medical staff can track the position of the first cannula inlet 2 and the second cannula outlet 11 by tracking the position of the radiopaque marker 15. In alternate embodiments, the present invention may include different means to help the medical staff track the positioning of the present invention within the body of the patient.

### EXEMPLARY METHOD OF THE PRESENT INVENTION

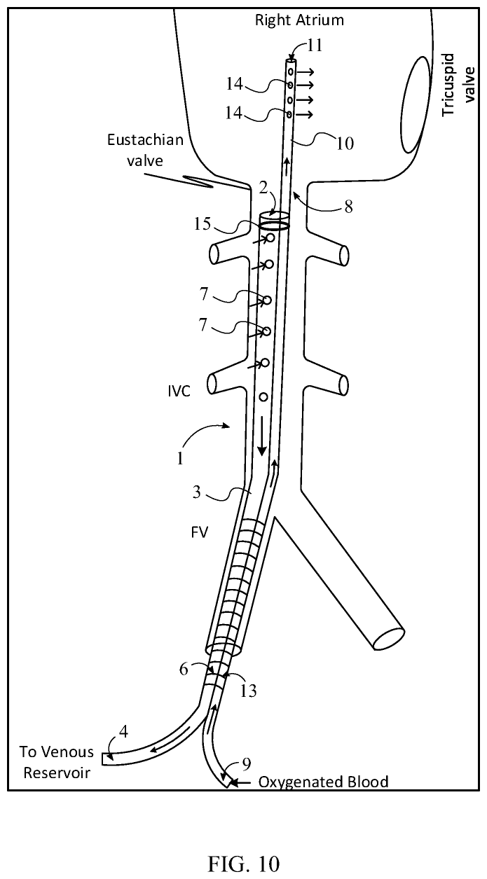

The double-lumen percutaneous femoral cannula for ECMO is designed so that the present invention can be inserted into the femoral vein to avoid the technical challenge of having to insert the large cannula into the internal jugular vein under the adverse circumstances, as can be seen in FIG. 10. Once inserted, the oxygenated blood may be pumped back into the right atrium facing the tricuspid valve through the at least one second cannula 8. Further, the deoxygenated blood may be drained from the inferior vena cava into the membrane oxygenator outside the body through the at least one first cannula 1.

Further, when a patient may need more support of oxygenation than veno-veno ECMO, the present invention may be reconfigured to veno-arterial ECMO. The present invention may be advanced with the at least one second cannula 8 positioned in the superior vena cava, and the at least one first cannula 1 positioned in the right atrium and inferior vena cava. The second cannula inlet 9 the first cannula outlet 4 may be connected to the appropriate connectors (V connector and A connector, respectively), then through Y connector to drain into the venous drainage of the membrane oxygenator. A separate arterial line may be inserted into the femoral artery for the return of oxygenated blood from the membrane oxygenator.

The present invention is also easier to be deployed into the patient to initiate veno-veno ECMO when patients are extremely ill. The patients may be on a rota-prone bed, the head of the bed elevated to optimize oxygenation, the head of the patient strapped, and on high pressure support ventilation. Once the veno-veno ECMO is initiated and patients stabilized, some of the patients may be able to be transferred to the intensive care unit of the hospital or transferred to tertiary care centers for further management. Some patients may require higher blood flow. In this situation, since the patient is stabilized, another catheter can be inserted into the internal jugular vein or subclavian vein under more control situation. In other situation when veno-veno ECMO support is not adequate and patients need veno-arterial ECMO, the present invention may serve for drainage for the veno-arterial ECMO. The at least one second cannula 8 is positioned in the superior vena cava and the at least one first cannula 1 in the inferior vena cava. A separate cannula may be inserted into the femoral artery to complete the circuit of the veno-arterial ECMO. The present invention is useful to begin the veno-veno ECMO in community hospital settings. Further, the patient may be transferred safely to as tertiary center (or hospital setting) for further management. Even in a tertiary hospital setting, the present invention is useful for the management of acute respiratory failure patient. Further, for the patients that require additional support, a separate catheter can be inserted into the subclavian or the internal jugular vein under a controlled environment. If the patient requires longer-term support, the present invention can always be changed to the internal jugular vein under more ideal situation.

For insertion of the present invention, a trocar may be required that can be inserted into the double lumen cannula. The trocar may be made of silastic material, like the present invention. The trocar may also include a central channel for the guide wires and another channel to accommodate the at least one second cannula 8. The trocar should be at 10 cm longer than the present invention. Further, the femoral vein may be punctured with or with the aid of ultrasound locating device. Once the femoral vein is punctured, a guidewire is then inserted and advanced into the femoral vein, iliac vein, inferior vena cava, and right atrium. A small skin incision is made adjacent to the guidewire. This will make stretching of the incision site with a dilator easier. The track between the skin and the femoral vein is gradually dilated by succeeding size of the dilator, threading the dilator through the guidewire inserted earlier, meaning from 6 Fr, 10 Fr, 14 Fr, and gradually up to 24 Fr if a 27 Fr cannula is to be used for the veno-veno ECMO. Local pressure has to be applied on the femoral vein when the dilators are removed, and larger dilators inserted over the guidewire to prevent blood loss during the changing of the dilators. Further, the length of the present invention to be inserted may be estimated by measuring from the femoral puncture site to the middle of the sternum. A silk suture is then tied on the first cannula body 3 to mark the approximate length to be inserted. Further, the guidewire is passed into the central channel of the trocar that was in the present invention. The trocar with the present invention is then advanced into the femoral vein, iliac vein, the inferior vena cava, and the right atrium. To keep the orientation of the present invention whereby the second cannula outlet 11 is facing the tricuspid valve, two black lines may be incorporated on the first cannula body 3. During insertion, the two black lines should be facing 12 O'clock position.

Once the present invention is inserted until the silk marker, the trocar is removed. A vascular clamp is then applied on the present invention. The V-connector is then connected to the first cannula outlet 4 and the A-connector to the second cannula inlet 9. The ECMO machine is then primed and ready. Further, a tubing of the ECMO is divided. The 0.75 in tubing where deoxygenated blood is drained into the membrane Oxygenator is connected to the V connector. The 0.75 in tubing where oxygenated blood is to be pumped back to the patient is connected to the A connector. Careful attention must be paid to ensure that there is no air bubble in the circuit during this process. Once the connector of the ECMO circuit is accomplished, the clamps on the present invention are removed. Veno-veno ECMO support can then be initiated. In some patients where veno-veno ECMO is inadequate to support oxygenation of the patient, veno-arterial (VA) ECMO is required. In this situation, the present invention can be advanced for another 5 cm so that the second cannula outlet 11 is in the lumen of the superior vena cava. Both the first cannula outlet 4 and the second cannula inlet 9 are connected with a Y-connector, and then to the venous end of the VA ECMO circuit. A separate arterial cannula is inserted into the femoral artery for return of oxygenated blood from the membrane oxygenator.

Although the invention has been explained in relation to its preferred embodiment, it is to be understood that many other possible modifications and variations can be made without departing from the spirit and scope of the invention.

What is claimed is:

1. A double-lumen percutaneous femoral cannula for extracorporeal membrane oxygenation (ECMO) comprising:
at least one first cannula;
at least one second cannula;
the at least one first cannula comprising a first cannula inlet, a first cannula body, and a first cannula outlet;
the at least one second cannula comprising a second cannula inlet, a second cannula body, and a second cannula outlet;
the second cannula inlet laterally and hermetically traversing into the first cannula body, adjacent to the first cannula outlet;
the second cannula body traversing through the first cannula body and out of the first cannular inlet;
the second cannula body being tangentially mounted within the first cannula body; and,
the second cannula outlet being positioned external to the first cannula body, offset to the first cannula inlet.

2. The double-lumen percutaneous femoral cannula for ECMO as claimed in claim 1, wherein an inner diameter of the first cannular body is larger than an outer diameter of the second cannular body.

3. The double-lumen percutaneous femoral cannula for ECMO as claimed in claim 1, wherein the first cannula outlet and the second cannula inlet are oriented away from each other.

4. The double-lumen percutaneous femoral cannula for ECMO as claimed in claim 1 comprising:
the at least one first cannula further comprising a plurality of first corrugated rings;
the plurality of first corrugated rings being laterally integrated into the first cannula body;
the plurality of first corrugated rings being positioned offset from the first cannula inlet; and,
the plurality of first corrugated rings being positioned offset from the first cannula outlet.

5. The double-lumen percutaneous femoral cannula for ECMO as claimed in claim 4 comprising:
the at least one second cannula further comprising a plurality of second corrugated rings;
the plurality of second corrugated rings being laterally integrated into the second cannula body;
the plurality of second corrugated rings being positioned offset from the second cannula inlet;
the plurality of second corrugated rings being positioned offset from the second cannula outlet; and,
the plurality of second corrugated rings being positioned within the first cannula body, adjacent to the plurality of first corrugated rings.

6. The double-lumen percutaneous femoral cannula for ECMO as claimed in claim 1 comprising:
the at least one first cannula further comprising a plurality of first orifices;
the plurality of first orifices laterally traversing into the first cannula body, adjacent to the first cannula inlet; and,
the plurality of first orifices being positioned offset from the second cannula body about the first cannula body.

7. The double-lumen percutaneous femoral cannula for ECMO as claimed in claim 1 comprising:
the at least one second cannula further comprising a plurality of second orifices;
the plurality of second orifices laterally traversing into the second cannula body, adjacent to the second cannula outlet; and,
the plurality of second orifices being positioned external to the first cannula body.

8. The double-lumen percutaneous femoral cannula for ECMO as claimed in claim 1 comprising:
a radiopaque marker; and,
the radiopaque marker being integrated into the first cannula body, adjacent to the first cannula inlet.

9. A double-lumen percutaneous femoral cannula for extracorporeal membrane oxygenation (ECMO) comprising:
at least one first cannula;
at least one second cannula;
a radiopaque marker;
the at least one first cannula comprising a first cannula inlet, a first cannula body, and a first cannula outlet;
the at least one second cannula comprising a second cannula inlet, a second cannula body, and a second cannula outlet;
the second cannula inlet laterally and hermetically traversing into the first cannula body, adjacent to the first cannula outlet;
the second cannula body traversing through the first cannula body and out of the first cannular inlet;
the second cannula body being tangentially mounted within the first cannula body;
the second cannula outlet being positioned external to the first cannula body, offset to the first cannula inlet; and,
the radiopaque marker being integrated into the first cannula body, adjacent to the first cannula inlet.

10. The double-lumen percutaneous femoral cannula for ECMO as claimed in claim 9, wherein an inner diameter of the first cannular body is larger than an outer diameter of the second cannular body.

11. The double-lumen percutaneous femoral cannula for ECMO as claimed in claim 9, wherein the first cannula outlet and the second cannula inlet are oriented away from each other.

12. The double-lumen percutaneous femoral cannula for ECMO as claimed in claim 9 comprising:
the at least one first cannula further comprising a plurality of first corrugated rings;
the at least one second cannula further comprising a plurality of second corrugated rings;
the plurality of first corrugated rings being laterally integrated into the first cannula body;
the plurality of first corrugated rings being positioned offset from the first cannula inlet;
the plurality of first corrugated rings being positioned offset from the first cannula outlet;
the plurality of second corrugated rings being laterally integrated into the second cannula body;
the plurality of second corrugated rings being positioned offset from the second cannula inlet;
the plurality of second corrugated rings being positioned offset from the second cannula outlet; and,
the plurality of second corrugated rings being positioned within the first cannula body, adjacent to the plurality of first corrugated rings.

13. The double-lumen percutaneous femoral cannula for ECMO as claimed in claim 9 comprising:
the at least one first cannula further comprising a plurality of first orifices;
the plurality of first orifices laterally traversing into the first cannula body, adjacent to the first cannula inlet; and,
the plurality of first orifices being positioned offset from the second cannula body about the first cannula body.

14. The double-lumen percutaneous femoral cannula for ECMO as claimed in claim 9 comprising:
the at least one second cannula further comprising a plurality of second orifices;
the plurality of second orifices laterally traversing into the second cannula body, adjacent to the second cannula outlet; and,
the plurality of second orifices being positioned external to the first cannula body.

15. A double-lumen percutaneous femoral cannula for extracorporeal membrane oxygenation (ECMO) comprising:
at least one first cannula;
at least one second cannula;
a radiopaque marker;
the at least one first cannula comprising a first cannula inlet, a first cannula body, a first cannula outlet, and a plurality of first corrugated rings;
the at least one second cannula comprising a second cannula inlet, a second cannula body, and a second cannula outlet;
the second cannula inlet laterally and hermetically traversing into the first cannula body, adjacent to the first cannula outlet;
the second cannula body traversing through the first cannula body and out of the first cannular inlet;
the second cannula body being tangentially mounted within the first cannula body;
the second cannula outlet being positioned external to the first cannula body, offset to the first cannula inlet;
the radiopaque marker being integrated into the first cannula body, adjacent to the first cannula inlet;
the plurality of first corrugated rings being laterally integrated into the first cannula body;
the plurality of first corrugated rings being positioned offset from the first cannula inlet; and,
the plurality of first corrugated rings being positioned offset from the first cannula outlet.

16. The double-lumen percutaneous femoral cannula for ECMO as claimed in claim 15, wherein an inner diameter of the first cannular body is larger than an outer diameter of the second cannular body.

17. The double-lumen percutaneous femoral cannula for ECMO as claimed in claim 15, wherein the first cannula outlet and the second cannula inlet are oriented away from each other.

18. The double-lumen percutaneous femoral cannula for ECMO as claimed in claim 15 comprising:
the at least one second cannula further comprising a plurality of second corrugated rings;
the plurality of second corrugated rings being laterally integrated into the second cannula body;
the plurality of second corrugated rings being positioned offset from the second cannula inlet;
the plurality of second corrugated rings being positioned offset from the second cannula outlet; and,
the plurality of second corrugated rings being positioned within the first cannula body, adjacent to the plurality of first corrugated rings.

19. The double-lumen percutaneous femoral cannula for ECMO as claimed in claim 15 comprising:
the at least one first cannula further comprising a plurality of first orifices;
the plurality of first orifices laterally traversing into the first cannula body, adjacent to the first cannula inlet; and,
the plurality of first orifices being positioned offset from the second cannula body about the first cannula body.

20. The double-lumen percutaneous femoral cannula for ECMO as claimed in claim 15 comprising:
the at least one second cannula further comprising a plurality of second orifices;
the plurality of second orifices laterally traversing into the second cannula body, adjacent to the second cannula outlet; and,
the plurality of second orifices being positioned external to the first cannula body.
